(19) United States
(12) Patent Application Publication
(10) Pub. No.: US 2024/0278033 A1
(43) Pub. Date: Aug. 22, 2024

(54) BIOCOMPATIBLE ELECTROMAGNETIC (BIOELECTROMAGNETIC) APPARATUS

(71) Applicant: George Roth, Markham (CA)

(72) Inventor: George Roth, Markham (CA)

(21) Appl. No.: 18/589,235

(22) Filed: Feb. 27, 2024

Related U.S. Application Data
(63) Continuation-in-part of application No. PCT/CA22/51300, filed on Aug. 26, 2022.
(60) Provisional application No. 63/237,608, filed on Aug. 27, 2021.

(51) Int. Cl. A61N 2/02; A61N 2/00

(52) U.S. Cl. CPC A61N 2/02; A61N 2/006

(57) ABSTRACT

A signal generator is configured to generate a signal to be converted into a biocompatible electromagnetic signal. A coil assembly is configured to receive the signal, and to produce a biocompatible electromagnetic frequency. A magnet assembly is positioned in relation to the coil assembly to augment the range of transmission of the biocompatible electromagnetic field (signal) in the environment. In this manner, the biocompatible electromagnetic signal is broadcast into the environment of a living organism, including a human, and imparts at least some beneficial influence to the living organism.

### CROSS-REFERENCE

This application claims priority from U.S. Provisional Patent Application No. 63/237,608 filed Aug. 27, 2021, which is incorporated by reference as if fully set forth herein.

### TECHNICAL FIELD

This disclosure relates to the technical field of an apparatus configured to broadcast a specified electromagnetic signal or field into an environment, and tuning the signal or field to be beneficial to humans within range of the field, and methods thereof.

### BACKGROUND

The human body (or any other living entities, such as animals, etc.) generates and emanates a biocompatible electromagnetic field (and/or signals). Bioelectromagnetics or biocompatible electromagnetics are terms used to describe the interaction between the biological electromagnetic fields produced by living cells of the tissues of the body (living or biological entities, etc.), the natural background electromagnetic field generated by the earth (which is inherently biocompatible) and artificial sources of non-biocompatible electromagnetic fields generated by manufactured devices such as mobile phones, wireless routers, smart meters, etc.

Non-biocompatible electromagnetic fields have been shown to cause abnormal physiology of organic life forms, including the human body, in certain studies. The application of a biocompatible electromagnetic field is thought to optimize normal physiology. Known bioelectromagnetic-based devices, such as pulsed fields or magnetic fields, may provide biocompatible electromagnetic fields. Biocompatible electromagnetic fields tend to mitigate, or compensate for the adverse effects caused by non-biocompatible electromagnetic fields to the body. It is thought that biocompatible bioelectromagnetic fields or signals may support general well-being and protect against the potentially deleterious effects of non-biocompatible electromagnetic fields in certain susceptible individuals. It is known that this benefit can extend to plant and animal life as well as humans.

### BRIEF SUMMARY

A problem associated with known bioelectromagnetic-based devices is that they require the individual to remain confined to a relatively small area, or to wear the device directly on their body, in order to receive any mitigation benefit. It is desirable to have devices that permit the individual to be less confined and experience relatively more movement (preferably more freedom of movement) while they receive a beneficial biocompatible bioelectromagnetic field.

It is therefore desirable to have devices that are able to broadcast the electromagnetic field, in the sense that the electromagnetic field has strength over a wider spatial area.

It is also desirable that such a device be capable of broadcasting the strengthened electromagnetic field at specific frequencies which are considered biocompatible in nature. In a preferred embodiment, it is desirable that the signal be similar in nature to that emitted by the Earth's magnetic core, the so-called Terrestrial Magnetic Field (TMF). In another preferred embodiment, the signal can be modulated to simulate the diurnal cycle of the Terrestrial Magnetic Field, or to support various desired physiological outcomes.

In accord with the present invention there is provided an apparatus for use with a plant, animal, or human being positioned in an environment, the apparatus comprising: a first digital signal generator, a first digital analog converter, a first amplifier, and a first coil assembly; the apparatus being configured so the first digital signal generator generates a signal, which passes through the first digital analog converter and the first amplifier and through the first coil assembly, the first coil assembly thus generating a first electromagnetic field, and a first magnet assembly located proximate the first coil assembly; where the first electromagnetic field has a first biocompatible signal frequency.

In an aspect of the invention, the first biocompatible signal frequency is at least one Schumann frequency. In another aspect, the first biocompatible signal frequency is 8+/−0.5 Hz. In still another aspect, the magnet assembly is a disc with alternating polarities, where one side of the disc is abutting the coil assembly.

In another aspect, there is provided a second digital signal generator, a second digital analog converter, a second amplifier, and a second coil assembly; the apparatus being configured so the second digital signal generator generates a signal, which passes through the second digital analog converter and the second amplifier and through the second coil assembly, the second coil assembly thus generating a second electromagnetic field, and a second magnet assembly located proximate the second coil assembly; where the second electromagnetic field has a second biocompatible signal frequency.

In still another aspect, the second biocompatible signal frequency is at least one Schumann frequency. In another aspect, the second biocompatible signal frequency is 14+/−0.5 Hz. In another aspect, the second biocompatible signal frequency is 20+/−0.5 Hz. In another aspect, the first digital signal generator and the second digital signal generator are the same digital signal generator. In still another aspect, the first digital signal generator comprises a microcontroller in communication with a read time clock, the microcontroller comprising an internal oscillator and a counter.

In another aspect, the microcontroller is in communication with a keypad and a display, and the keypad can be used to control the operation of the microcontroller. In still another aspect, the microcontroller is configured so the biocompatible signal frequencies can be changed at specific times.

In accord with the present invention, there is provided an apparatus for use with a plant, animal, or human being positioned in an environment, the apparatus comprising: a signal generator being configured to generate a signal; a coil assembly including a spirally-coiled wire being configured to receive the signal from the signal generator, and broadcast an electromagnetic field; and a magnet assembly located proximate the coil assembly; where the signal has a biocompatible signal frequency.

In an aspect of the invention, the biocompatible signal frequency is at least one of the Schumann frequencies. In another aspect, the biocompatible signal frequency is 8+/−0.5 Hz. In still another aspect, the magnet assembly is a disc having elements with alternating polarities, where one side of the disc is abutting the coil assembly. In still another aspect, there is an auxiliary coil assembly connected to the signal generator configured to emit an auxiliary electromagnetic field. In another aspect, there is an antenna assembly connected to the signal generator configured to emit an electromagnetic field. In yet another aspect, the magnet assembly includes a magnet array having magnet sections being positioned within the magnet array.

In another aspect, the magnet sections are concentrically positioned, one within the other within a neighboring magnet section; and said magnet sections include: a first magnet section being positioned within a first central zone of the magnet array; and a second magnet section defining a second central zone configured to receive the first magnet section, and the second magnet section surrounds an outer peripheral edge of the first magnet section; and a third magnet section defining a third central zone configured to receive the second magnet section, and the third magnet section surrounds the outer peripheral edge of the second magnet section; and a fourth magnet section defining a fourth central zone configured to receive the third magnet section, and the fourth magnet section surrounds the outer peripheral edge of the third magnet section.

In accord with the present invention, there is provided a wireless router configured to operate at a Schumann frequency. In an aspect of this invention, the Schumann frequency is in the range of approximately 3-300 Hz.

In accord with the present invention, there is provided a wireless router incorporating an apparatus comprising: a first digital signal generator, a first digital analog converter, a first amplifier, and a first coil assembly; the apparatus being configured so the first digital signal generator generates a signal, which passes through the first digital analog converter and the first amplifier and through the first coil assembly, the first coil assembly thus generating a first electromagnetic field, and a first magnet assembly located proximate the first coil assembly; where the first electromagnetic field has a first biocompatible signal frequency; and configured to operate at a Schumann frequency. In an aspect of this invention, the Schumann frequency is in the range of approximately 3-300 Hz.

In accord with the present invention, there is provided a wireless router incorporating an apparatus comprising: a signal generator being configured to generate a signal; a coil assembly including a spirally-coiled wire being configured to receive the signal from the signal generator, and broadcast an electromagnetic field; and a magnet assembly located proximate the coil assembly; where the signal has a biocompatible signal frequency; and configured to operate at a Schumann frequency. In an aspect of the invention, the Schumann frequency is in the range of approximately 3-300 Hz.

In accord with the present invention, there is provided a method of improving the functioning of a living organism, comprising exposing the living organism to an electromagnetic field generated by an apparatus comprising: a first digital signal generator, a first digital analog converter, a first amplifier, and a first coil assembly; the apparatus being configured so the first digital signal generator generates a signal, which passes through the first digital analog converter and the first amplifier and through the first coil assembly, the first coil assembly thus generating a first electromagnetic field, and a first magnet assembly located proximate the first coil assembly; where the first electromagnetic field has a first biocompatible signal frequency. In an aspect of the invention, the first biocompatible signal frequency is at least one Schumann frequency.

In accord with the present invention, there is provided a method of improving the functioning of a living organism, comprising exposing the living organism to an electromagnetic field generated by an apparatus comprising: a signal generator being configured to generate a signal; a coil assembly including a spirally-coiled wire being configured to receive the signal from the signal generator, and broadcast an electromagnetic field; and a magnet assembly located proximate the coil assembly; where the signal has a biocompatible signal frequency; and configured to operate at a Schumann frequency. In an aspect of the invention, the first biocompatible signal frequency is at least one Schumann frequency.

In accord with the present invention, there is provided an apparatus for use with a plant, animal, or human being positioned in an environment, the apparatus comprising: a digital signal generator including a microcontroller, a digital analog converter, an amplifier, and a coil assembly; a magnet assembly comprising a disc having separate magnetic elements with alternating polarities, the magnet assembly located proximate the coil assembly, where one side of the disc is abutting the coil assembly; the apparatus being configured so the digital signal generator generates a signal, which passes through the digital analog converter and the amplifier and through the coil assembly, the coil assembly thus generating an electromagnetic field, and the intensity of the electromagnetic field being controlled by the microcontroller; where the electromagnetic field has a biocompatible signal frequency chosen from the set of Schumann frequencies.

In an aspect of the present invention, the set of Schumann frequencies is 8+/−0.5 Hz, 14+/−0.5 Hz, 21+/−0.5 Hz. In another aspect, the set of Schumann frequencies is the delta band ranging from about 0.5 to about 3.5 Hertz, the theta band from about 4 to about 7 Hertz, the alpha band from about 8 to about 13 Hertz, and the beta band from about 14 to about 30 Hertz. In another aspect, the microcontroller is in communication with a real-time clock wherein the microcontroller is configured to allow the user to schedule specific biocompatible signal frequencies and intensities to be broadcast at specific times to simulate the diurnal cycle of the Terrestrial Magnetic Field. In another aspect, the microcontroller is in communication with a real-time clock wherein the microcontroller is configured to allow the user to schedule specific biocompatible signal frequencies to be broadcast at specific times to be customized to the desired sleep-wake cycles of the individual. IN yet another aspect, the schedule broadcasts biocompatible signal frequencies 3 Hz+/−0.5 Hz and 8 Hz+/−0.5 Hz during a first period and broadcasts biocompatible signals 8 Hz+/−0.5 Hz, 14 Hz+/−0.5 Hz and 20 Hz+/−0.5 Hz during a second period. In another aspect, the schedule cycles through biocompatible signal frequencies 3 Hz+/−0.5 Hz and 8 Hz+/−0.5 Hz during a first period and cycles through biocompatible signals 8 Hz+/−0.5 Hz, 14 Hz+/−0.5 Hz and 20 Hz+/−0.5 Hz during a second period. In another aspect, the electromagnetic field can be modulated at certain frequencies, intensities and times to support various desired physiological outcomes, including 40 Hz+/−0.5 Hz. In still another aspect, the magnet sections are concentrically positioned, one within the other; and said magnet sections include: a first magnet section being positioned within a first central zone of the magnet array; and a second magnet section defining a second central zone configured to receive the first magnet section, and the second magnet section surrounds an outer peripheral edge of the first magnet section; and a third magnet section defining a third central zone configured to receive the second magnet section, and the third magnet section surrounds the outer peripheral edge of the second magnet section; and a fourth magnet section defining a fourth central zone configured to receive the third magnet section, and the fourth magnet section surrounds the outer peripheral edge of the third magnet section.

In accord with the present invention, there is provided a method of improving the functioning of a living organism, comprising exposing the living organism to an electromagnetic field generated by an apparatus comprising: a digital signal generator including a microcontroller, a digital analog converter, an amplifier, and a coil assembly; a magnet assembly comprising a disc having separate magnetic elements with alternating polarities, the magnet assembly located proximate the coil assembly, where one side of the disc is abutting the coil assembly; the apparatus being configured so the digital signal generator generates a signal, which passes through the digital analog converter and the amplifier and through the coil assembly, the coil assembly thus generating an electromagnetic field, and the intensity of the electromagnetic field being controlled by the microcontroller; where the electromagnetic field has a biocompatible signal frequency chosen from the set of Schumann frequencies.

In an aspect of the present invention, the set of Schumann frequencies is 8+/−0.5 Hz, 14+/−0.5 Hz, 21+/−0.5 Hz. In another aspect, the set of Schumann frequencies is the delta band ranging from about 0.5 to about 3.5 Hertz, the theta band from about 4 to about 7 Hertz, the alpha band from about 8 to about 13 Hertz, and the beta band from about 14 to about 30 Hertz. In another aspect, the microcontroller is in communication with a real-time clock wherein the microcontroller is configured to allow the user to schedule specific biocompatible signal frequencies and intensities to be broadcast at specific times to simulate the diurnal cycle of the Terrestrial Magnetic Field. In another aspect, the microcontroller is in communication with a real-time clock wherein the microcontroller is configured to allow the user to schedule specific biocompatible signal frequencies to be broadcast at specific times to be customized to the desired sleep-wake cycles of the individual. IN yet another aspect, the schedule broadcasts biocompatible signal frequencies 3 Hz+/−0.5 Hz and 8 Hz+/−0.5 Hz during a first period and broadcasts biocompatible signals 8 Hz+/−0.5 Hz, 14 Hz+/−0.5 Hz and 20 Hz+/−0.5 Hz during a second period. In another aspect, the schedule cycles through biocompatible signal frequencies 3 Hz+/−0.5 Hz and 8 Hz+/−0.5 Hz during a first period and cycles through biocompatible signals 8 Hz+/−0.5 Hz, 14 Hz+/−0.5 Hz and 20 Hz+/−0.5 Hz during a second period. In another aspect, the electromagnetic field can be modulated at certain frequencies, intensities and times to support various desired physiological outcomes, including 40 Hz+/−0.5 Hz. In still another aspect, the magnet sections are concentrically positioned, one within the other; and said magnet sections include: a first magnet section being positioned within a first central zone of the magnet array; and a second magnet section defining a second central zone configured to receive the first magnet section, and the second magnet section surrounds an outer peripheral edge of the first magnet section; and a third magnet section defining a third central zone configured to receive the second magnet section, and the third magnet section surrounds the outer peripheral edge of the second magnet section; and a fourth magnet section defining a fourth central zone configured to receive the third magnet section, and the fourth magnet section surrounds the outer peripheral edge of the third magnet section.

### BRIEF DESCRIPTION OF THE DRAWINGS

The non-limiting embodiments may be more fully appreciated by reference to the following detailed description of the non-limiting embodiments when taken in conjunction with the accompanying drawings, in which:

FIG. 1 depicts a schematic view of an embodiment of an apparatus for use with an individual;

FIG. 2 depicts a schematic view of an alternative embodiment of the apparatus of FIG. 1;

FIG. 3 depicts a schematic view of an alternative embodiment of the apparatus of FIG. 1;

FIG. 4 depicts a schematic view of an alternative embodiment of the apparatus of FIG. 1;

FIG. 5 depicts a schematic view of a preferred embodiment of the apparatus of FIG. 1;

FIG. 6 depicts a schematic view of a preferred embodiment of the apparatus of FIG. 1;

FIG. 8 depicts a schematic view of a preferred embodiment of the apparatus of FIG. 1;

FIG. 9 and FIG. 10 depict a front view (FIG. 9) and a cross-sectional view (FIG. 10) of embodiments of a magnet assembly for use with the apparatus of FIGS. 1-8;

FIG. 11, FIG. 12, FIG. 13, FIG. 14 and FIG. 15 depict front views (FIG. 11 and FIG. 12), a perspective view (FIG. 13), a front view (FIG. 14), and a perspective view (FIG. 15) of embodiments of a magnet assembly for use with the apparatus of FIGS. 1-8;

FIG. 16, FIG. 17, FIG. 18, FIG. 19 and FIG. 20 depict front views (FIG. 16 and FIG. 17), a perspective view (FIG. 18), and front views (FIG. 19 and FIG. 20) of embodiments of a coil assembly for use with the apparatus of FIGS. 1-8;

FIG. 21 depicts an observation table associated with the apparatus of FIG. 1 and FIG. 2; and

FIG. 22 depicts a method of testing for physiological effects associated with the apparatus of FIG. 1 and FIG. 2 listed in FIG. 21.

### LISTING OF REFERENCE NUMERALS USED IN THE DRAWINGS

signal generator 100
output terminals (101A, 101B)
digital signal generator 103
signal 104
signal 104a
coil assembly 200
coil assembly 200a
coil assembly 200b
electromagnetic field 204
electromagnetic field 204a
electromagnetic field 204b
disc 206
spirally-coiled wire 208
planar surface 210
first coil terminal 211
second coil terminal 212
outer peripheral edge 214
magnet assembly 300
magnet assembly 300a
magnet assembly 300b
magnet array 301
disc 303
magnetic field lines 304
magnetic field lines 304a
magnetic field lines 304b
radially-extending gap zone 306
magnet sections (308A, 308B, 308C, 308D, 308E)
linear magnet sections (310A, 310B, 310C, 310D)
magnet sections (312A, 312B, 312B, 312C, 312D)
auxiliary coil assembly 400
auxiliary electromagnetic field 404
antenna assembly 500
antenna-based electromagnetic field 504
housing assembly 601
housing assembly 602
digital analog converter 603
digital analog converter 603a
digital analog converter 603b
amplifier 605
amplifier 605a
amplifier 605b
housing terminals 611
non-biocompatible electromagnetic signal 700
biocompatible electromagnetic signal 800
individual 900
1000
real time clock 1000
battery 1002
microcontroller 1004
internal oscillator 1006
counter 1008
keypad 1010
display 1012
digital analog converter 1014
amplifier 1016
coil assembly 1018
length (of coil assembly) 1019
magnet assembly 1020
digital analog converter 1022
amplifier 1024
coil assembly 1026
length (of coil assembly) 1028
magnet assembly 1030
hand 2002
thigh 2004
pressure 2006
multimeter set to resistance (Ω) 2008

### DETAILED DESCRIPTION

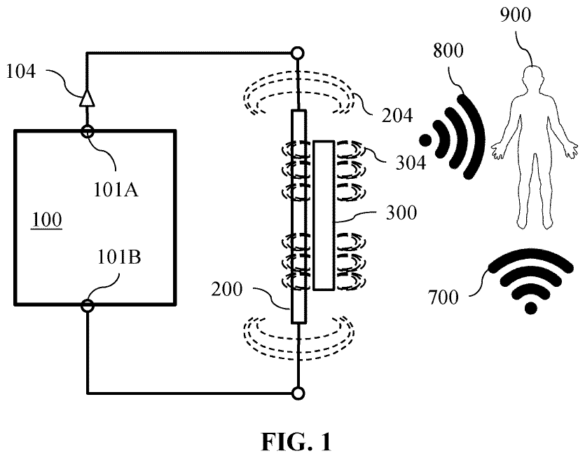

FIG. 1 depicts a schematic view of an embodiment of an apparatus to project a strengthened electromagnetic field. As shown in FIG. 1, the apparatus is for use with an individual 900.

Turning to FIG. 1, there is a signal generator 100 configured to generate an electric signal 104. A coil assembly 200 is connected via input terminals 101A and 101B to the signal generator 100. Coil assembly 200 is proximate magnet assembly 300. In accordance with a preferred embodiment, the coil assembly 200 includes a spirally-coiled wire 208 (or an equivalent thereof), including the spirally coiled wires illustrated in FIGS. 19 and 20, configured to receive the signal 104.

In operation, the signal 104 passes through coil assembly 200 and generates a magnetic field 204. The magnet assembly 300 is configured to emit the magnetic field lines 304. The magnet assembly 300 is positioned in close proximity to, or in contact with the coil assembly 200 in such a way that the electromagnetic field 204 and the magnetic field lines 304 interact with or augment each other to produce an electromagnetic field 800. In a preferred embodiment, close proximity to means within 5 mm.

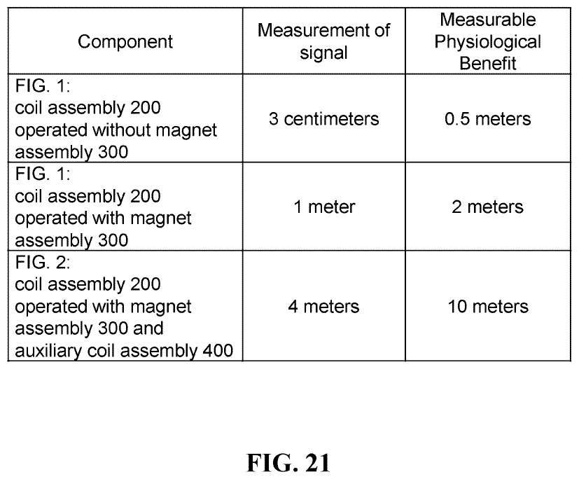

Referring to the embodiment as depicted in FIG. 1 but without magnetic coil 300, measuring equipment (such as the REED Model GU-3001 ELECTROMAGNETIC FIELD (EMF) METER, manufactured by REED INSTRUMENTS) was used to detect the presence of electromagnetic signal activity (that is, measure the amount or magnitude of the electromagnetic signal 800). Signal detection was measured within a distance of about three (3) centimeters from the outer surface of the coil assembly 200. Beyond this distance of three centimeters, electromagnetic signal activity was not detected. This is more fully described below in association with FIG. 21

Returning to FIG. 1, there is an individual 900, who as part of their normal life is exposed to non-biocompatible electromagnetic signal(s) 700. By adjusting the frequency of current or signal 104, the frequency of electromagnetic field 800 can be tuned to turn electromagnetic signal 800 into a biocompatible electromagnetic signal 800.

Biocompatible terrestrial magnetic field (TMF) includes Schumann resonance frequencies. Exposure to Schumann resonance frequencies is considered to be beneficial to the health and well-being of people and other life forms. Schumann resonance frequencies are part of the background stationary electromagnetic noise that propagates in the cavity between the earth's surface and the lower boundary of the ionosphere. The peak intensity of the Schumann resonance frequencies includes about 8 Hertz (˜7.83 Hz), along with its harmonics with lower intensity at about 14, 20, 26, 33, 39, and 45 Hertz due to frequency-related, ionospheric propagation loss.

The peak Schumann resonance frequencies undergo a moderate diurnal variation (24-hour day/night variations) of approximately plus or minus 0.5 Hertz. The first four modes of the Schumann resonance frequencies happen to be within the frequency range of the first four electroencephalogram bands (EEG bands): the delta band ranges from about 0.5 to about 3.5 Hertz, the theta band from about 4 to about 7 Hertz, the alpha band from about 8 to about 13 Hertz, and the beta band from about 14 to about 30 Hertz). This is why it is believed that the Schumann frequencies are beneficial to supporting neurological function. The frequency of about 8 Hertz (plus or minus 0.5 Hertz) Schumann resonance frequencies (background signals) may vary depending on diurnal variations (24-hour day/night variations) and seasonal variations (spring, summer, etc.) in response to stochastic (random) redistribution of electric activity over the globe and resultant changes in the local height of the ionosphere at the respective observational sites.

In a preferred embodiment, the frequency of the electromagnetic signal 800 is about 8 Hz, in which case electromagnetic signal 800 is a biocompatible electromagnetic signal 800. In a preferred embodiment, the frequency of the electromagnetic signal 800 is about 8 Hz+/−0.5 Hz, in which case electromagnetic signal 800 is a biocompatible electromagnetic signal 800. In a preferred embodiment, the frequency of the electromagnetic signal 800 is set to match the Schumann resonance frequencies.

Referring to the embodiment as depicted in FIG. 1, but without magnetic coil 300, the frequency of the electromagnetic field was tuned to 8 Hz, and measuring equipment (such as the REED Model GU-3001 ELECTROMAGNETIC FIELD (EMF) METER, manufactured by REED INSTRUMENTS) was used to detect the presence of electromagnetic signal activity. A signal was detected within a distance of about three (3) centimeters from the outer surface of the coil assembly 200. However, a biological benefit (physical benefit) to individual 900 was confirmed up to approximately 0.5 meters between the outer surface of the coil assembly 200 and individual 900. For the case where individual 900 is positioned or placed further than 0.5 meters away from the coil assembly 200, physiological testing of individual 900 demonstrated no apparent biological benefit. It is presumed, though not confirmed, that in the 3 cm to 0.5 meter range, the signal 800 was too weak to be detected by the instruments used in this experiment, but were still of sufficient strength to benefit the individual 900. This is more fully described below in association with FIG. 21

It will be appreciated that precise measurements and observations will depend on the geometries of the coil assembly 200, the type of measuring equipment used, etc.

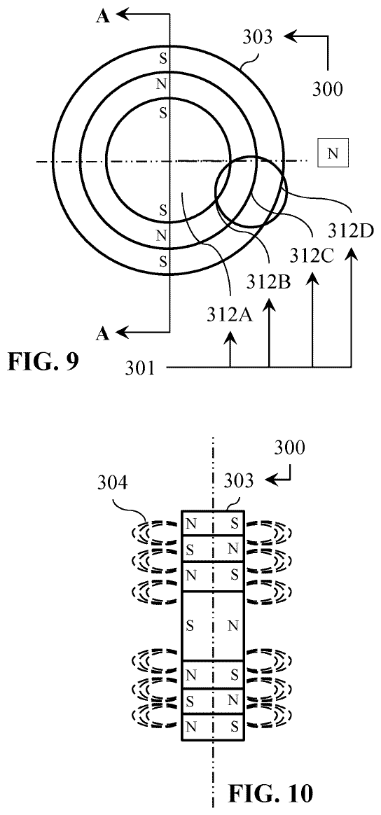

Turning to FIG. 1, the magnet assembly 300 is configured to be positioned proximate to the coil assembly 200. A preferred embodiment of the magnet assembly 300 is illustrated in FIG. 9. Turning to FIG. 9, the magnet assembly 300 includes a magnet array 301 that has a plurality of magnet sections 312A, 312B, 312C, 312D. configured to be positioned proximate to the coil assembly 200.

Returning to the embodiment as depicted in FIG. 1, with the inclusion of the magnet assembly 300, measuring equipment was used to detect the biocompatible electromagnetic signal 800. The relevant signal was detected within a distance of about one (1) meter from the combination of the coil assembly 200 and the magnet assembly 300. Beyond this distance of one meter, electromagnetic signal activity was not detected by the measuring equipment. With the addition of the magnet assembly 300, a biological benefit to individual 900 was confirmed for a distance of up to approximately two (2) meters between the outer surface of the coil assembly 200 and the individual 900. For the case where individual 900 is positioned or placed further than two meters away from the combination of the coil assembly 200 and the magnet assembly 300, physiological testing of individual 900 demonstrated no biological benefit. Again, it is presumed though not confirmed that in the 1 m to 2 m range, the signal 800 was too weak to be detected by the instruments used in this experiment, but were still of sufficient strength to benefit the individual 900. This is more fully described below in association with FIG. 21.

It is expected, but not confirmed through testing, that the apparatus will still provide benefits if the spirally-coiled wire 208 in coil assembly 200 is substituted with a non-spiral wire coil.

Based on our evaluation, we have determined that there is a physiological benefit to a combined constant and variable magnetic field—the constant field is provided by the magnet assembly 300, and the variable field is provided by the coil assembly 200 sourced by the electronic components of the device.

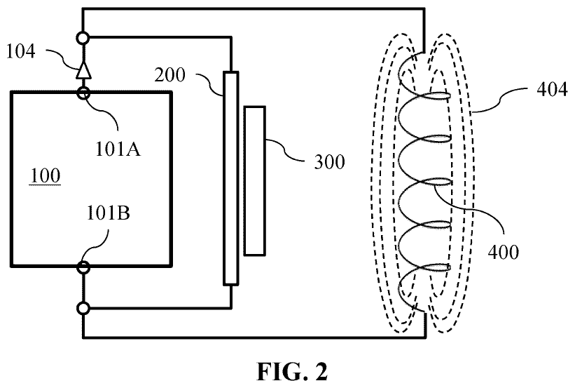

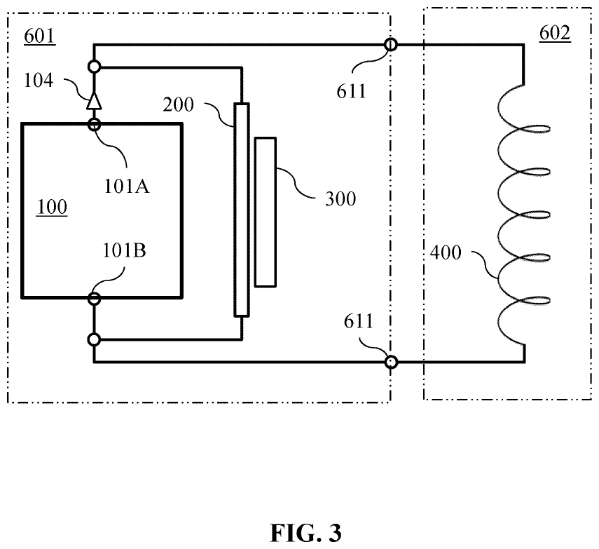

FIG. 2 and FIG. 3 depict schematic views of further embodiments of the apparatus of FIG. 1.

Turning to FIG. 2, there is an auxiliary coil assembly 400 added to the apparatus illustrated in FIG. 1. The auxiliary coil assembly 400 is connected to the output terminals 101A and 101B of signal generator 100. The auxiliary coil assembly 400 is configured to emit an auxiliary electromagnetic field 404. The auxiliary electromagnetic field 404 interacts with the electromagnetic field 204 magnetic field lines 304 to augment electromagnetic field 800. In this way, individual 900 may be advantageously positioned at a more distant location from coil assembly 200 and still experience electromagnetic field 800 (such as a position located even further away from the coil assembly 200 in comparison to the arrangement of FIG. 1).

By tuning the frequency of electromagnetic field 800 to the Schumann resonance frequencies, electromagnetic field 800 becomes a biocompatible electromagnetic field 800.

Referring to the embodiment as depicted in FIG. 2, with the addition of the auxiliary coil assembly 400, measuring equipment (such as the REED Model GU-3001 ELECTROMAGNETIC FIELD (EMF) METER, manufactured by REED INSTRUMENTS) was used to detect electromagnetic signal activity of the biocompatible electromagnetic signal 800. Signal 800 was detected within a distance of about four (4) meters from the combination of the coil assembly 200 and the magnet assembly 300. Beyond this distance of four meters, electromagnetic signal activity was not detected (measured) by the measuring equipment. A biological benefit to individual 900 was confirmed for a distance of about ten (10) meters between the coil assembly 200 and the individual 900. For the case where individual 900 is positioned (placed) further than ten meters away from the combination of the coil assembly 200 and the magnet assembly 300, physiological testing of individual 900 demonstrated no biological benefit. Again, it is presumed, though not confirmed, that in the 4 m to 10 m range, the signal 800 was too weak to be detected by the instruments used in this experiment, but were still of sufficient strength to benefit the individual 900. This is more fully described below in association with FIG. 21.

It will be appreciated that precise measurements and observations will depend on the geometries of the coil assembly 200, the magnet assembly 300, the auxiliary coil assembly 400, the type of measuring equipment used, etc.

The auxiliary coil assembly 400 may be housed in a different housing assembly than the rest of the apparatus. Turning to the embodiment depicted in FIG. 3, the signal generator 100, the coil assembly 200, and the magnet assembly 300 are positioned in a first housing assembly 601. The auxiliary coil assembly 400 is positioned in a second housing assembly 602. The housing assembly 601 includes spaced-apart housing terminals 611. The housing terminals 611 are electrically coupled to the output terminals 101A, 101B of the signal generator 100. The housing terminals 611 are configured to be electrically coupled to the input terminals of the auxiliary coil assembly 400. It is expected that this configuration will result in an electromagnetic field that covers more area than the apparatus of FIG. 1.

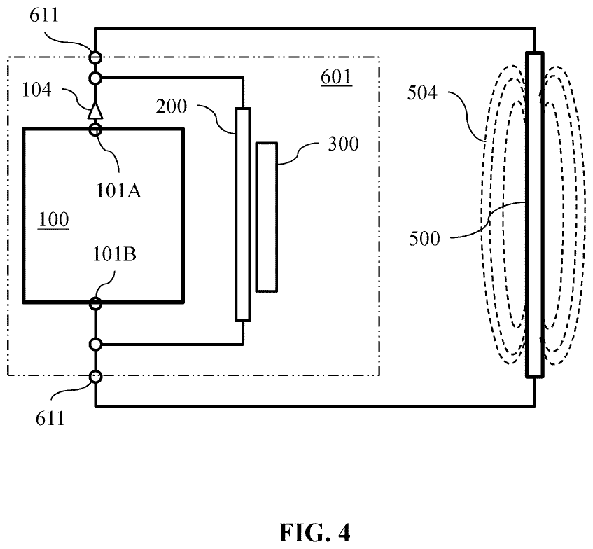

FIG. 4 depicts a schematic view of an alternative embodiment of the apparatus of FIG. 1. Turning to FIG. 4, there is added an antenna assembly 500. The antenna assembly 500 is configured to emit an antenna-based electromagnetic field 504. The antenna assembly 500 includes input terminals. The input terminals of the antenna assembly 500 are configured to be electrically connected (coupled) to the output terminals 101A, 101B of the signal generator 100. The antenna assembly 500 is configured to be in signal communication with the signal generator 100 (when the antenna assembly 500 is electrically connected or coupled to the output terminals (101A, 101B) of the signal generator 100). Referring to the embodiment as depicted in FIG. 4, the signal generator 100, the coil assembly 200, and the magnet assembly 300 are (preferably) positioned in the housing assembly 601. The antenna assembly 500 is positioned outside of the housing assembly 601.

It is expected that the antenna-based electromagnetic field 504 will interact with the electromagnetic field 204 and magnetic field lines 304 to further augment or strengthen electromagnetic field 800. In this way, individual 900 may be advantageously positioned at a more distant location from coil assembly 200 and still experience electromagnetic field 800 (such as a position located even further away from the coil assembly 200 in comparison to the arrangement of FIG. 1). The individual 900 may be positioned at an even more convenient location, or the individual may be able to move around more freely (compared to the case for the first position) while receiving the electromagnetic signal (in comparison to the embodiment of FIG. 1).

The antenna assembly 500 is expected to further extend the distance between the individual and the coil assembly 200 and the magnet assembly 300. Referring to the embodiment as depicted in FIG. 4 (with the addition of the antenna assembly 500), measuring equipment has not yet been used to measure the presence of electromagnetic signal activity (of the biocompatible electromagnetic signal 800) in the same way as was done in connection with FIGS. 1 and 2. This measurement is expected to further extend the distance between the coil assembly 200 and the individual 900 compared to the embodiments of FIG. 1. With the addition of the antenna assembly 500, a biological benefit (physical benefit) to individual 900 has not yet been confirmed, in the same way, as that confirmed in connection with FIG. 1. However, it is expected that individual 900 may experience at least some biological benefit at a further extended range between individual 900 and the coil assembly 200. The determination may depend on the specific configuration of the antenna assembly 500, etc. It will be appreciated that the measurements and observations will depend on the geometries of the coil assembly 200, the magnet assembly 300, the antenna assembly 500 (without the auxiliary coil assembly 400 installed), and the measuring equipment used, etc.

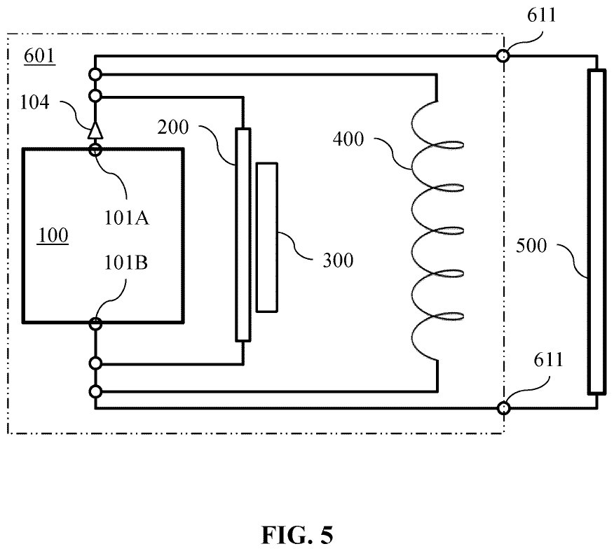

It will be appreciated by a person skilled in the art that the embodiments described above in FIGS. 2, 3 and 4 may be combined with improved effect. FIG. 5 depicts a schematic view of an embodiment of the apparatus of FIG. 1 that incorporates both an auxiliary antenna 500 and an auxiliary coil assembly 400. Turning to FIG. 5, the auxiliary coil assembly 400 and the antenna assembly 500 are configured to be in signal communication with the signal generator 100. The strength of (bio)compatible electromagnetic signal 800 may be further increased through the combination of electromagnetic fields 504, 404, 304 and 204.

Referring to the embodiment as depicted in FIG. 5, with the addition of the auxiliary coil assembly 400 and the antenna assembly 500, measuring equipment has not yet been used to detect (measure) the presence of electromagnetic signal activity (of the biocompatible electromagnetic signal 800) in the same way as that with FIG. 1 or FIG. 2. The result of these measurements is expected to further extend the distance between the coil assembly 200 and the individual 900 in comparison to the embodiments of FIG. 1, FIG. 2, or FIG. 3. With the addition of the auxiliary coil assembly 400 and the antenna assembly 500, a biological benefit to individual 900 has not yet been confirmed, as determined in connection with FIG. 1, FIG. 2, or FIG. 3. The result of this determination is that individual 900 is expected to experience at least some biological benefit at a further extended range between individual 900 and the coil assembly 200. The determination may be based on the specific configuration of the antenna assembly 500. It will be appreciated that the measurements and observations will depend on the geometries of the coil assembly 200, the magnet assembly 300, the auxiliary coil assembly 400, the antenna assembly 500, and the type of measuring equipment used, etc.

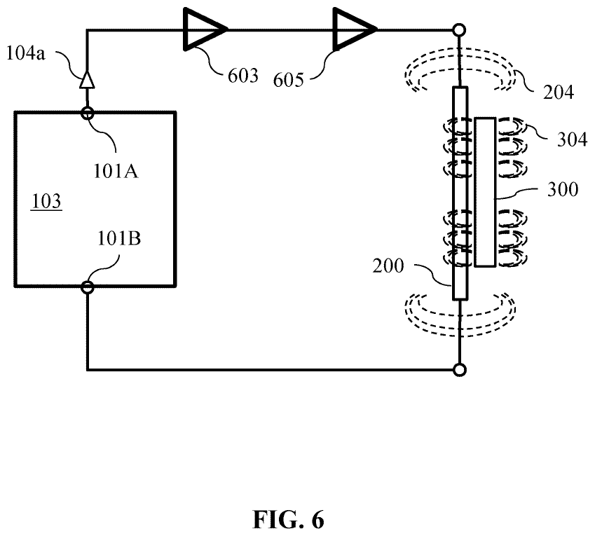

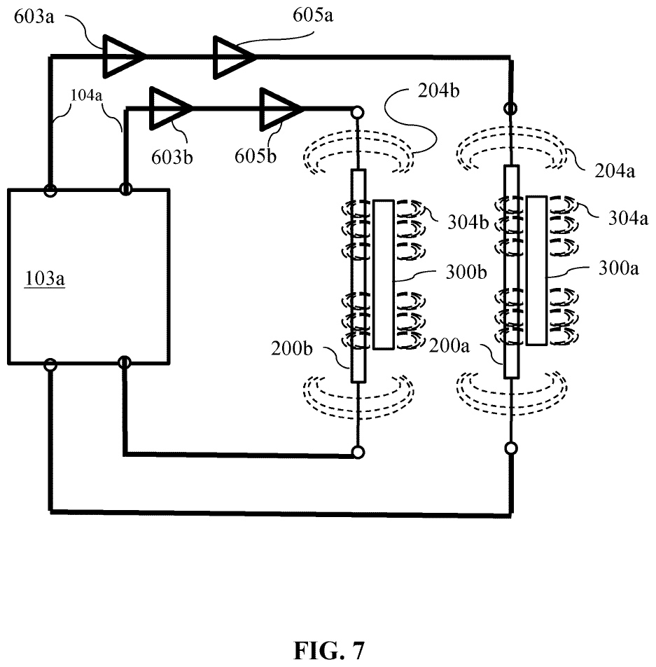

A preferred embodiment of the inventive equipment is illustrated in FIG. 6. Turning to FIG. 6, there is a digital signal generator 103 configured to generate a digital signal 104a. The digital signal 104a generated by digital signal generator 103 passes through a digital analog converter 603 and an amplifier 605 before flowing to coil assembly 200, which is proximate to magnet assembly 300. Amplifier 605 can be set to create a stronger signal to coil assembly 200 and thus stronger electromagnetic fields 204 and 304.

The coil assembly 200 and magnetic array 300 may be combined to strengthen the resulting electromagnetic fields. This may be seen in the preferred embodiment in FIG. 7. Turning to FIG. 7, there is a digital signal generator 103a configured to generate a digital signal 104b. The digital signal 104b generated by digital signal generator 103 passes through digital analog converters 603a and 603b and amplifiers 605a and 605b before flowing to coil assemblies 200a and 200b, which are proximate to magnet assemblies 300a and 300b. Amplifiers 605a and 605 b can be set to create a stronger signal to coil assemblies 200a and 200b and thus stronger electromagnetic fields 204a and 204b and 304a and 304b.

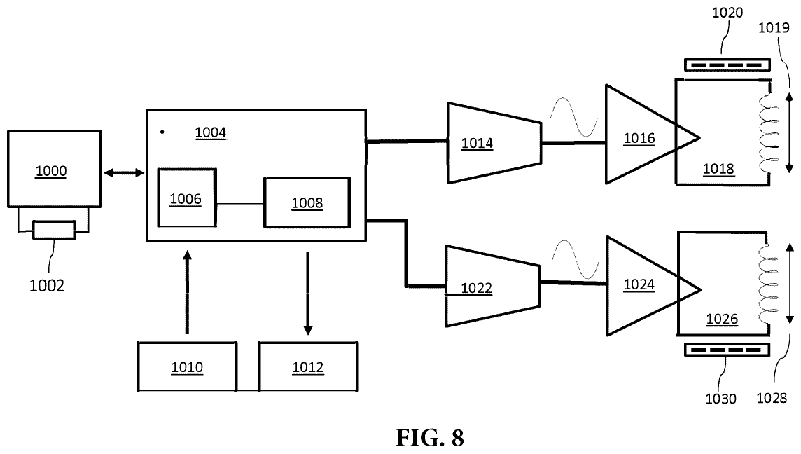

A still more preferred embodiment is illustrated in FIG. 8. Turning to FIG. 8, there is a real time clock 1000 connected to a battery 1002. The clock 1000 is connected to a microcontroller 1004 which incorporates an internal oscillator 1006 and a counter 1008. The microcontroller can be controlled by a user through keypad 1010, with the preferred settings shown on display 1012. The signal generated by microcontroller 1004 is fed through digital analog converter 1014 and amplifier 1016 and then through coil assembly 1018. Coil assembly 1018 has a length 1019, and magnet assembly 1020 is placed proximate to or abutting one end of coil assembly 1018. In a preferred embodiment, proximate means within 5 mm. The signal generated by microcontroller 1004 is also fed through digital analog converter 1022 and amplifier 1024 and then through coil assembly 1026. Coil assembly 1026 has a length 1028, and magnet assembly 1030 is placed proximate to or abutting one end of coil assembly 1028. In a preferred embodiment, proximate means within 5 mm. In one preferred embodiment, internal oscillator 1006 is set to 8 MHz, the frequency generated in coil assembly 1018 is approximately 8 Hz+/−0.5 Hz. This is the most dominant TMF during the night. In another embodiment, the frequency generated in coil assembly 1026 can be set to broadcast frequencies other than 8 Hz+/−0.5 Hz that are associated with the range of Schumann frequencies. In a specific embodiment, the frequencies generated in the coil assembly 1026 are between 3-300 Hz. In a specific embodiment, the frequencies generated in coil assembly 1026 are 14 Hz+/−0.5 Hz and 20 Hz+/−0.5 Hz, which are part of the TMF typically emitted during daytime.

In a preferred embodiment, the microcontroller 1004 is Microchip™ part no. PIC24FJ64GA705T-I/M4. In a preferred embodiment, the digital analog converters 1014 and 1022 are Microchip™ part no. MCP47CVB02-E/MF. In a preferred embodiment, the amplifiers 1016 and 1024 are Diodes™ part no. PAM8406DR. In a preferred embodiment, the coil assemblies 1918 and 1026 are Coilcraft™ part no. AST1236-T. In a preferred embodiment, the magnet assemblies 1918 and 1026 are Alternating Polity Concentric Magnets (APCM) from Niiomed™.

In a preferred embodiment, the signal 104 is a sinusoidal or simulated sinusoidal signal, with maximum current of 1 Ampere, midrange current of 500 milliampere and a minimum current of 50 milliampere.

In a further embodiment, microcontroller 1004 is programmable by the user to vary the intensity and frequency of the signals output by coil assemblies 1018 and 1028 over time. In a particular embodiment, the microcontroller 1004 is programmed to vary the signals output by coil assemblies 1018 and 1028 over time so that coil 1028 generates additional signal of frequencies in the range of 3-300 Hz. In a particular embodiment, the additional signal of frequencies is approx. 14 Hz+/−0.5 Hz and 20 Hz+/−0.5 Hz during daytime hours, 8 Hz+/−0.5 Hz at night.

FIG. 9 and FIG. 10 depict a front view (FIG. 9) and a cross-sectional view (FIG. 10) of embodiments of a magnet assembly 300 for use with the apparatus of FIGS. 1 to 8. The cross-sectional view of FIG. 7 is taken along a cross-sectional line A-A of FIG. 6.

The use of Schumann frequencies can also be used to create a biocompatible wireless router. Wireless routers presently operate at 2.4 GHz or 6 GHZ. By using the devices and embodiments disclosed in this application, and adding information to the signal, a wireless router can be designed that will cover an acceptable physical distance and can transmit data at the Schumann frequencies in the range of approximately 3-300 Hz. In a particular embodiment, frequencies used are 8 Hz+/−0.5 Hz, 14 Hz+/−0.5 Hz and 20 Hz+/−0.5 Hz.

Turning to FIG. 9, the magnet assembly 300 preferably includes a magnet array 301. The magnet array 301 has a plurality of magnet sections 312A, 312B, 312C, 312D positioned within the magnet array 301. It is believed that the magnet array 301 may be substituted with a non-array of magnetic elements and still operate desirably. In a further preferred embodiment, the plurality of magnet sections 312A, 312B, 312C, 312D are concentrically positioned, one within the other (within a neighboring magnet section). The magnet section 312A is positioned within a central zone of the magnet array 301. The magnet section 312B defines a central zone configured to receive the magnet section 312A, and the magnet section 312B surrounds the outer peripheral edge of the magnet section 312A. The magnet section 312C defines a central zone configured to receive the magnet section 312B, and the magnet section 312C surrounds the outer peripheral edge of the magnet section 312B. The magnet section 312D defines a central zone configured to receive the magnet section 312C, and the magnet section 312D surrounds the outer peripheral edge of the magnet section 312C.

The magnet array 301 may have two or more magnet sections. The magnet array 301 may have a circular shape or an elliptical shape.

Referring to the embodiment as depicted in FIG. 9, the polarity of the magnet sections 312A, 312B, 312C, 312D are preferably configured to attract each neighboring magnet member. This is done in such a way that the magnet array 301 becomes magnetically bonded together by mutual attraction. It will be appreciated that additional options may be used to enhance the interconnectedness of the magnet sections 312A, 312B, 312C, 312D as might be required.

In one preferred embodiment, referring to the embodiment as depicted in FIG. 9, the magnet assembly 300 includes any array of magnets as disclosed and described in U.S. Pat. No. 9,943,699 (Entitled: THERAPEUTIC MAGNET APPARATUS; Inventor: James J. SOUDER; Granted: 2018 Apr. 17), which is incorporated by reference into this disclosure.

Referring to the embodiments as depicted in FIG. 9 and FIG. 10, the magnet assembly 300 preferably forms a disc 303 preferably having an outer diameter of about 3.5 centimeters and a thickness of about 0.15 centimeters. It will be appreciated that other dimensions may be used if tested and verified. The disc 303 has opposite facing sides. One side is configured to be positioned proximate to, or in an abutting relationship with, the coil assembly 200 (as depicted in FIG. 1). It will be appreciated that the magnet assembly 300 may have any suitable shape configured to cooperate with the coil assembly 200 (as depicted in FIG. 1).

In a preferred embodiment, the magnet assembly 300 in FIGS. 1 through 8 directly abuts the magnetic coil 200. In a preferred embodiment, the coil assembly 200 has the shape of a cylinder, and the magnet assembly 300 abuts the coil assembly 200 at one end of the coil assembly 200. In another preferred embodiment, the magnet assembly 300 in FIGS. 1 through 8 is within 5 mm of magnetic coil 200. In a preferred embodiment, the coil assembly 200 has the shape of a cylinder, and the magnet assembly 300 is within 5 mm of one end of the coil assembly 200.

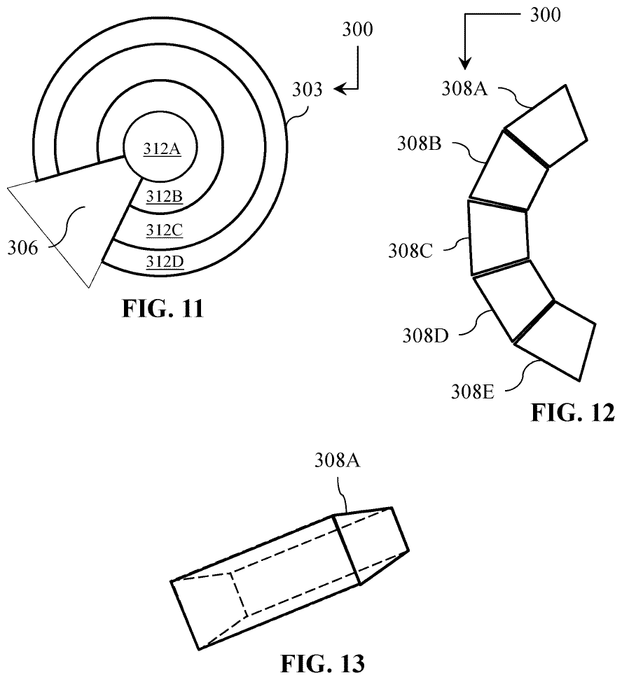

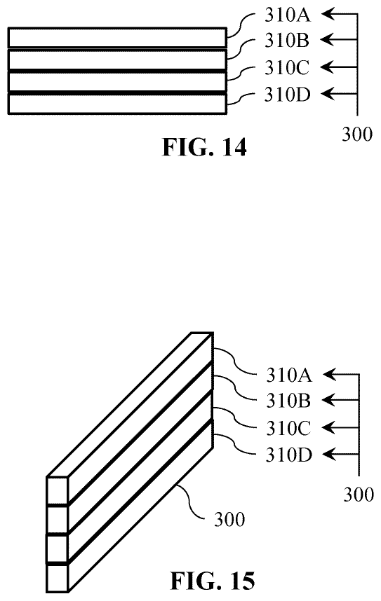

FIG. 11, FIG. 12, FIG. 13, FIG. 14 and FIG. 15 depict front views (FIG. 11 and FIG. 12), a perspective view (FIG. 13), a front view (FIG. 14), and a perspective view (FIG. 15) of embodiments of a magnet assembly 300 for use with the apparatus of FIGS. 1 through 8.

Referring to the embodiment as depicted in FIG. 11, the magnet assembly 300 defines a radially-extending gap zone 306. This arrangement is designed to augment the electromagnetic field 204 including the biocompatible electromagnetic field 800.

Referring to the embodiment as depicted in FIG. 12, the magnet assembly 300 includes angled magnet sections 308A, 308B, 308C, 308D, 308E having magnet sections positioned, one after the other, to form a C-shaped structure defining a radially-extending gap zone 306. This arrangement is designed to augment the electromagnetic field 204 including the biocompatible electromagnetic field 800.

Referring to the embodiment as depicted in FIG. 13, the angled magnet section 308A forms a trapezoidal formation. This arrangement is designed to augment the electromagnetic field 204 including the biocompatible electromagnetic field 800.

Referring to the embodiments as depicted in FIG. 14 and FIG. 15, the magnet assembly 300 includes a plurality of linear magnet sections (310A, 310B, 310C, 310D) positioned to form a linear wall-shaped structure. This arrangement is designed to augment the electromagnetic field 204 including the biocompatible electromagnetic field 800.

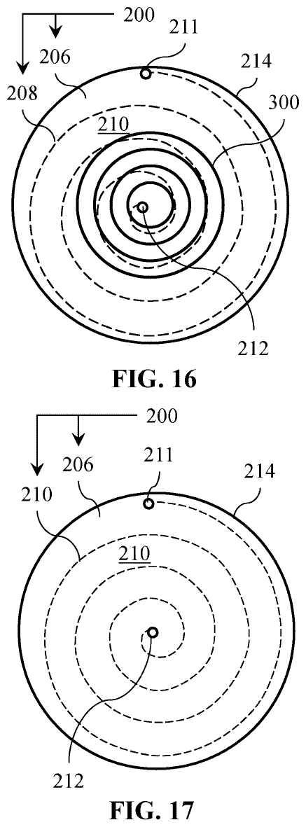

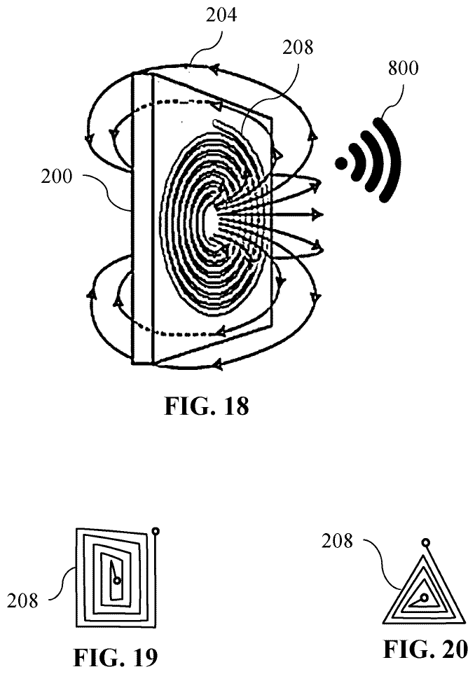

FIG. 16, FIG. 17, FIG. 18, FIG. 19 and FIG. 20 depict front views (FIG. 16 and FIG. 17), a perspective view (FIG. 18), and front views (FIG. 19 and FIG. 20) of embodiments of a coil assembly 200 for use with the apparatus of FIGS. 1 through 8.

Referring to the embodiments as depicted in FIG. 16 and FIG. 17, the coil assembly 200 includes a planar surface 210. The magnet assembly 300 is positioned proximate to the planar surface 210 of the coil assembly 200 as depicted in FIG. 16. The magnet assembly 300 is preferably affixed to and abuts the planar surface 210 of the coil assembly 200. The magnet assembly 300 is preferably positioned proximate to a central section of the planar surface 210 of the coil assembly 200 as depicted in FIG. 16. For clarity, FIG. 17 shows the coil assembly 200 from FIG. 16 without the magnet assembly 300.

Referring to the embodiments as depicted in FIG. 16 and FIG. 17, the coil assembly 200 includes a spirally-coiled wire 208. The coil assembly 200 includes (preferably) a disc 206 with a spirally-coiled wire 208 embedded therein. The disc 206 has, preferably, a diameter of about nine (9) centimeters, and a thickness of about one (1) millimeter. It will be appreciated that other dimensions of the disc 206 are possible (if tested and verified accordingly). The disc 206 is larger than the outer dimension of the magnet assembly 300. The disc 206 includes an outer peripheral edge 214 shaped (preferably or conveniently) to form a circle. It will be appreciated that the outer peripheral edge 214 may have any suitable shape. The outer peripheral edge 214 has an outer dimension that is larger than the outer dimension of the magnet assembly 300. The outer dimension of the magnet assembly 300 has (preferably) an outer dimension of about 3.5 centimeters and about 1.5 millimeters thick. It will be appreciated that other dimensions of the magnet assembly 300 are possible.

Referring to the embodiments as depicted in FIG. 16 and FIG. 17, the spirally-coiled wire 208 terminates at a first coil terminal 211 and a second coil terminal 212. The first coil terminal 211 is positioned at an outer peripheral portion (outer edge) of the planar surface 210. The second coil terminal 212 is positioned at a central portion of the planar surface 210. The spirally-coiled wire 208 (preferably) has a cross-sectional thickness of about one (1) millimeters. The spirally-coiled wire 208 (preferably) forms many spiraling turns as might be fitted to the coil assembly 200, depending on the geometries of the coil assembly 200 and the spirally-coiled wire 208.

Referring to the embodiment as depicted in FIG. 18, the spirally-coiled wire 208 is configured to form the electromagnetic field 204 (including the biocompatible electromagnetic signal 800) when the spirally-coiled wire 208 receives the signal 104 from the signal generator 100 (as depicted in FIG. 1). The spirally-coiled wire 208 forms the electromagnetic field 204, including the biocompatible electromagnetic signal 800, once the spirally-coiled wire 208 receives the signal 104 from the signal generator 100.

Referring to the embodiments as depicted in FIG. 16, FIG. 17, FIG. 19 and FIG. 20, the spirally-coiled wire 208 forms a circular-shaped spiral formation (as depicted in FIG. 17). The spirally-coiled wire 208 forms a rectangular-shaped spiral formation (as depicted in FIG. 19). The spirally-coiled wire 208 forms a triangular-shaped spiral formation (as depicted in FIG. 20). It will be appreciated that the spirally-coiled wire 208 may form any suitably shaped spiral formation.

In one preferred embodiment, the signal can be modulated to simulate the diurnal cycle of the Terrestrial Magnetic Field, or to support various desired physiological outcomes. In a particular embodiment, the apparatus can generate frequencies of between 3 Hz+/−0.5 Hz and 8 Hz+/−0.5 Hz during the night, and 8 Hz+/−0.5 Hz, 14 Hz+/−0.5 Hz and 20 Hz+/−0.5 Hz during the day. In a further embodiment, the frequencies can be on a cycling program, in the day cycling between 8 Hz+/−0.5 Hz, 14 Hz+/−0.5 Hz and 20 Hz+/−0.5 Hz, and in the night cycling between 3 Hz+/−0.5 Hz and 8 Hz+/−0.5 Hz.

The cycling of frequencies does not need to match the diurnal cycle of the TMF (which depends upon the geographic location and time of year), and instead can be customized to the desired sleep-wake cycles of the individual. For example, a shift worker who works at night may customize the device to a different sleep-wake cycle compared to a shift worker who works during the day.

In another embodiment, the device is configured to cycle through two or more frequencies. In another embodiment, the device can be programmed to cycle between customized specific frequencies to reflect the user's needs.

In another embodiment, additional frequencies may be provided in order to provide specific physiological effects, including 40 Hz+/−0.5 Hz to support certain neurological conditions such as Alzheimer's disease and support meditation practice.

In a preferred embodiment, the magnet assembly is a disc having separate magnetic elements with alternating polarities (as seen in FIG. 9, magnet assembly 301, having an array of magnets, which is more fully described in U.S. Pat. No. 9,943,699 (Titled: THERAPEUTIC MAGNET APPARATUS; Inventor: James J. SOUDER; Granted: 2018 Apr. 17). This magnet assembly has been shown through testing to have the unique property of generating a series of magnetic gradients, which amplifies the output of the coil assembly to a greater degree as compared to the output of the same coil assembly when applying a standard bipolar magnet of the same strength. Using this magnet assembly (a disc having separate magnetic elements with alternating polarities) is more practical in that it allows for much greater amplification than could be achieved with a reasonably-sized standard bipolar magnet.

### Test Results

FIG. 21 depicts a table of test results associated with the apparatus of FIG. 1 and of FIG. 2. Turning to FIG. 21, the first row contains results for the apparatus in FIG. 1 without the use of magnetic coil assembly 300; the second row contains results for the apparatus in FIG. 1 with the use of magnetic coil assembly 300; and the third row contains results for the apparatus in FIG. 2 with the use of magnetic coil assembly 300 and auxiliary coil assembly 400.

The equipment used in the test was as illustrated in FIGS. 1 and 2, using standard, off-the-shelf coils, magnets and signal generators. The electromagnetic field or signal was measured using standard equipment, such as a REED Model GU-3001 ELECTROMAGNETIC FIELD (EMF) METER, manufactured by REED INSTRUMENTS.

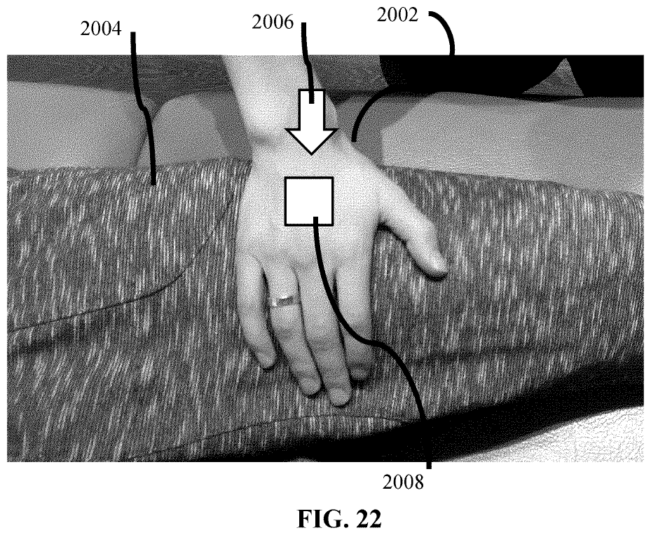

The physiological effects or benefits were determined using human test subjects. The testing procedure is illustrated in FIG. 22. Turning to FIG. 22, myofascial resistance or tone was measured according to the depth of deflection obtained with the subject at rest (with the hand 2002 resting upon thigh 2004 within an environment standardized for temperature, lighting and the presence of ambient electromagnetic fields (replicating a commercial office environment with typically generated Wi-Fi, smartphone and other potential sources). A digital pressure gauge, consisting of a resistive pressure sensor coupled with a multimeter set to resistance (Ω) 2008, was used as an interface to standardize the amount of pressure 2006 applied (standardized to 100+/−50Ω). The amount of tissue deflection was recorded with and without the influence of the biocompatible electromagnetic device and field. A difference of 1 cm or more of deflection was assigned a positive result for tissue response to the applied field.

What is claimed is:

1. An apparatus for use with a plant, animal, or human being positioned in an environment, the apparatus comprising:
i. a digital signal generator including a microcontroller, a digital analog converter, an amplifier, and a coil assembly;
ii. a magnet assembly comprising a disc having separate magnetic elements with alternating polarities, the magnet assembly located proximate the coil assembly, where one side of the disc is abutting the coil assembly;
iii. the apparatus being configured so the digital signal generator generates a signal, which passes through the digital analog converter and the amplifier and through the coil assembly, the coil assembly thus generating an electromagnetic field, and the intensity of the electromagnetic field being controlled by the microcontroller;
iv. where the electromagnetic field has a biocompatible signal frequency chosen from the set of Schumann frequencies.

2. The apparatus of claim 1, where the set of Schumann frequencies is 8+/−0.5 Hz, 14+/−0.5 Hz, 21+/−0.5 Hz.

3. The apparatus of claim 1, where the set of Schumann frequencies is the delta band ranging from about 0.5 to about 3.5 Hertz, the theta band from about 4 to about 7 Hertz, the alpha band from about 8 to about 13 Hertz, and the beta band from about 14 to about 30 Hertz.

4. The apparatus of claim 1, where the microcontroller is in communication with a real-time clock wherein the microcontroller is configured to allow the user to schedule specific biocompatible signal frequencies and intensities to be broadcast at specific times to simulate the diurnal cycle of the Terrestrial Magnetic Field.

5. The apparatus of claim 1, where the microcontroller is in communication with a real-time clock wherein the microcontroller is configured to allow the user to schedule specific biocompatible signal frequencies to be broadcast at specific times to be customized to the desired sleep-wake cycles of the individual.

6. The apparatus of claim 4, where the schedule broadcasts biocompatible signal frequencies 3 Hz+/−0.5 Hz and 8 Hz+/−0.5 Hz during a first period and broadcasts biocompatible signals 8 Hz+/−0.5 Hz, 14 Hz+/−0.5 Hz and 20 Hz+/−0.5 Hz during a second period.

7. The apparatus of claim 4, where the schedule cycles through biocompatible signal frequencies 3 Hz+/−0.5 Hz and 8 Hz+/−0.5 Hz during a first period and cycles through biocompatible signals 8 Hz+/−0.5 Hz, 14 Hz+/−0.5 Hz and 20 Hz+/−0.5 Hz during a second period.

8. The apparatus of claim 4, wherein the electromagnetic field can be modulated at certain frequencies, intensities and times to support various desired physiological outcomes, including 40 Hz+/−0.5 Hz.

9. The apparatus of claim 1, wherein said magnet sections are concentrically positioned, one within the other, and said magnet sections include:
i. a first magnet section being positioned within a first central zone of the magnet array; and
ii. a second magnet section defining a second central zone configured to receive the first magnet section, and the second magnet section surrounds an outer peripheral edge of the first magnet section; and
iii. a third magnet section defining a third central zone configured to receive the second magnet section, and the third magnet section surrounds the outer peripheral edge of the second magnet section; and
iv. a fourth magnet section defining a fourth central zone configured to receive the third magnet section, and the fourth magnet section surrounds the outer peripheral edge of the third magnet section.

10. A method of improving the functioning of a living organism, comprising exposing the living organism to an electromagnetic field generated by an apparatus comprising:
i. a digital signal generator including a microcontroller, a digital analog converter, an amplifier, and a coil assembly;
ii. a magnet assembly comprising a disc having separate magnetic elements with alternating polarities, the magnet assembly located proximate the coil assembly, where one side of the disc is abutting the coil assembly;
iii. the apparatus being configured so the digital signal generator generates a signal, which passes through the digital analog converter and the amplifier and through the coil assembly, the coil assembly thus generating an electromagnetic field, and the intensity of the electromagnetic field being controlled by the microcontroller;
iv. where the electromagnetic field has a biocompatible signal frequency chosen from the set of Schumann frequencies.

11. The method of claim 10, where the set of Schumann frequencies is 8+/−0.5 Hz, 14+/−0.5 Hz, 21+/−0.5 Hz.

12. The method of claim 10, where the set of Schumann frequencies is the delta band ranging from about 0.5 to about 3.5 Hertz, the theta band from about 4 to about 7 Hertz, the alpha band from about 8 to about 13 Hertz, and the beta band from about 14 to about 30 Hertz.

13. The method of claim 10, where the microcontroller is in communication with a real-time clock wherein the microcontroller is configured to allow the user to schedule specific biocompatible signal frequencies and intensities to be broadcast at specific times to simulate the diurnal cycle of the Terrestrial Magnetic Field.

14. The method of claim 13, where the microcontroller is in communication with a real-time clock wherein the microcontroller is configured to allow the user to schedule specific biocompatible signal frequencies to be broadcast at specific times to be customized to the desired sleep-wake cycles of the individual.

15. The method of claim 13, where the schedule broadcasts biocompatible signal frequencies 3 Hz+/−0.5 Hz and 8 Hz+/−0.5 Hz during a first period and broadcasts biocompatible signals 8 Hz+/−0.5 Hz, 14 Hz+/−0.5 Hz and 20 Hz+/−0.5 Hz during a second period.

16. The method of claim 13 where the schedule cycles through biocompatible signal frequencies 3 Hz+/−0.5 Hz and 8 Hz+/−0.5 Hz during a first period and cycles through biocompatible signals 8 Hz+/−0.5 Hz, 14 Hz+/−0.5 Hz and 20 Hz+/−0.5 Hz during a second period.

17. The method of claim 13, wherein the electromagnetic field can be modulated at certain frequencies, intensities and times to support various desired physiological outcomes including 40 Hz+/−0.5 Hz.

18. The method of claim 10, wherein said magnet sections are concentrically positioned, one within the other, and said magnet sections include:
i. a first magnet section being positioned within a first central zone of the magnet array; and
ii. a second magnet section defining a second central zone configured to receive the first magnet section, and the second magnet section surrounds an outer peripheral edge of the first magnet section; and
iii. a third magnet section defining a third central zone configured to receive the second magnet section, and the third magnet section surrounds the outer peripheral edge of the second magnet section; and
iv. a fourth magnet section defining a fourth central zone configured to receive the third magnet section, and the fourth magnet section surrounds the outer peripheral edge of the third magnet section.
